(19) United States
(12) Patent Application Publication
(10) Pub. No.: US 2005/0197654 A1
(43) Pub. Date: Sep. 8, 2005

(54) Multiple section parenteral drug delivery apparatus

(76) Inventors: Carl F. Edman, San Diego, CA (US); Darrel D. Drinan, San Diego, CA (US); Robert P. Lackey, Carlsbad, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/009,548

(22) Filed: Dec. 9, 2004

Related U.S. Application Data
(60) Provisional application No. 60/529,162, filed on Dec. 12, 2003.

(51) Int. Cl. A61K 9/22

(52) U.S. Cl. 604/891.1

(57) ABSTRACT

The invention relates to a parenteral therapeutic agent delivery device. The therapeutic agent delivery device has a disposable section and an implant section suitable for long term implantation within the tissue of a subject. When necessary, the disposable section can be detached from the implant section, and a new disposable section can be attached. The disposable section may contain a reservoir containing the therapeutic agent, a pump for dispensing the therapeutic agent, controlling circuitry for regulating the dispensing of the therapeutic agent, and transceiver circuitry and an antenna for wireless communication with external devices.

### RELATED APPLICATIONS

This application claims priority under 35 U.S.C. § 119(e) to U.S. Provisional Application 60/529,162, filed on Dec. 12, 2003.

### BACKGROUND OF THE INVENTION

1. Field of the Invention

This invention relates to drug delivery apparatus having two major sections. Such apparatus are useful for long term administration of therapeutic agents in adjustable amounts or schedules.

2. Description of the Related Art

A number of devices have been described for the delivery of therapeutic agents, such as insulin, in a parenteral fashion. Such devices include the use of needles plus manual syringes, fully implanted systems which need to be periodically recharged with agents, microneedle based devices, or catheter-plus-pump systems. Each of these systems, while useful for certain applications, fails to provide a method of automatically delivering therapeutic agents over an extended period of time in a convenient and adjustable fashion.

For instance, Flaherty et al. (U.S. Pat. Nos. 6,656,158, 6,656,159, and 6,749,587) describe a low cost, remotely programmable device for the delivery of fluids, e.g. insulin, to patients. Such devices are described as being suitable for delivery systems utilizing needles or connected to infusion systems having skin penetrating cannula. In particular, U.S. Pat. No. 6,749,587 describes a modular infusion device consisting of a disposable portion and a reusable portion. The reusable portion contains the more expensive components, and the disposable portion contains a fluid reservoir and a transcutaneous patient access tool, such as a cannula for penetrating the skin of a patient. While this arrangement of components reduces the cost of the modular system, it does not provide the level of flexibility which may be required for certain applications, particularly those involving the delivery of multiple therapeutic agents. In addition, Flaherty does not provide a device suitable for long term parental implantation, as the transcutaneous patient access tool is located in the disposable portion.

Therefore, there remains a need to provide low cost, replaceable, drug delivery systems having a long term parenteral infusion device and a removable, replaceable adjustable reservoir device having pumping and communication ability.

### SUMMARY OF CERTAIN INVENTIVE ASPECTS

In an embodiment of the invention, there is a parenteral therapeutic agent delivery device comprising an access port comprising a parenteral fluid delivery location, an interior lumenal space, and a first connection point; a disposable section comprising a reservoir configured to hold a therapeutic agent, a pumping device, controlling circuitry to regulate delivery of the therapeutic agent, and a second connection point, configured to mate with the first connection point.

In a further embodiment of the invention, the disposable section additionally comprises transceiver circuitry, an antenna, and a power source, and the controlling circuitry is configured to utilize signals received via the antenna and the transceiver circuitry in regulating the delivery of the therapeutic agent.

In a further embodiment of the invention, the controlling circuitry is configured to transmit information regarding the delivery of the therapeutic agent via the transceiver circuitry and antenna.

In a further embodiment of the invention, the device additionally comprises sensors, and the controlling circuitry is configured to process signals received from the sensors, and utilize processed signals from the sensors in regulating the delivery of the therapeutic agent.

In another embodiment of the invention, there is a parenteral fluid delivery device comprising an access port and a disposable section, the access port being suitable for long term implantation within the tissue of a subject, wherein the access port is detachably coupled to the disposable section, wherein the access port comprises a connection point, a lumenal space in fluid communication with the connection point, and a biofluid head, the biofluid head configured for long term implantation by incorporating features promoting cellular ingrowth and inhibiting fibrous encapsulation of at least a portion of the biofluid head; and the disposable section comprises a reservoir configure to hold fluid, a pumping device, controlling circuitry to regulate delivery of the fluid, and a connection point.

This invention may be embodied in many different forms and should not be construed as being limited to the embodiments described above. Those skilled in the art will readily understand the basis of the invention as described by the embodiments.

### BRIEF DESCRIPTION OF THE DRAWINGS

FIG. 1—Generalized illustration of one embodiment of an access port plus disposable section.

FIG. 2—General illustration of access port features.

FIG. 3—Diagram of one embodiment of a biofluid head.

FIG. 4—Block diagram of one embodiment of controlling circuitry.

### DETAILED DESCRIPTION OF CERTAIN INVENTIVE EMBODIMENTS

The following description presents certain specific embodiments of the invention. However, the invention may be embodied in a multitude of different ways as defined and covered by the claims. In this description, reference is made to the drawings wherein like parts are designated with like numerals throughout.

As used herein, the term biofluids refers to fluids found in extracellular environments, e.g. interstitial fluid or cerebrospinal fluid, throughout the body of the subject which may contain a variety of materials, including but not limited to, proteins, hormones, nutrients, electrolytes, catabolic products, or introduced foreign substances.

As used herein, the term drug delivery platform (DDP) refers to a structure which comprises a disposable section and an implanted access port and will deliver defined volumes of drug upon command.

As used herein, the term disposable section refers to a replaceable or removable externally accessible component of the DDP.

As used herein, the term access port refers to a clinician inserted percutaneous component of the DDP.

As used herein, the phrase “long-term implantation” refers to implantation having duration of approximately 30 days or more.

As used herein, the term therapeutic agents refers to various compounds and materials, including, but not limited to: small molecular weight drugs; molecular scale sensing devices or materials; bioactive substances; enzymes; peptides, proteins; gene therapy agents; viral-based bio-agents; and/or micro- or nano-scale devices or materials. These materials and/or devices may be delivered for a variety of purposes, including, but not limited to: the relief of detected conditions; for preventative treatments; and as mobile sensors, detectors or other aids to diagnosis, treatment or measurement.

As used herein, the term Local Area Network (LAN) refers to a communication system providing bi-directional or unidirectional communication over short distances between two or more transceivers. Advantageous LANs employ radiofrequency-based communication. In the context of this invention, LANs may also employ, but are not limited to, optical or acoustic communication. As will be readily understood by a person skilled in the art, a LAN need not be a wireless network, although a wireless network advantageously allows communication with a transceiver attached to an ambulatory subject without the need for cumbersome wires.

Embodiments of the invention address the shortcomings mentioned above by providing methods and devices which allow continuous or periodic parenteral delivery of drugs or other therapeutic agents. An embodiment of this invention utilizes an apparatus referred to as a drug delivery platform (DDP). The DDP is intended to deliver drugs directly to locations beneath the skin, including but not limited to, subcutaneous, intramuscular, intravenous, intraperitoneal delivery, as well as to the cerebrospinal fluid. It comprises two or more major sections: one or more replaceable/removable disposable sections and a percutaneous access port.

In this embodiment, the disposable section is a user removable/replaceable unit intended to be removed and replaced periodically, e.g. about every 7-14 days, and is mounted on the outside of the skin and placed in fluid communication with the access port. The access port is a percutaneous device implanted through the skin which provides a long term (e.g. 30 days or more) port for subcutaneous drug delivery. In some advantageous embodiments, the implanted device is suitable for implantation for 90 days or more. As the drug solution is depleted, the entire disposable section may be removed and replaced with a new section containing additional drug solution. This platform may be operated either in continual or intermittent communication with one or more off-body devices. The devices themselves may be in communication with one or more remote data management systems.

This invention may include the use of one or more connecting points, which may include but are not limited to electrical, mechanical, optical or fluidic connecting points, between one or more disposable sections and an access. Within the disposable section, one or more therapeutic agent containment areas, e.g. reservoirs, and pump systems may be contained. In a preferred embodiment, a single disposable section has a single reservoir which contains a single therapeutic agent but in other embodiments, two or more reservoirs may be contained within a single disposable section. Such embodiments facilitate multidrug delivery through the same access port. Multidrug delivery may be made using the same delivery timing or rates for each drug to be delivered. Alternatively, each agent may be delivered separately with its own delivery schedule or rate. In yet other embodiments of the invention, one or more therapeutic agents or materials are combined into a mixture for co-administration through the access port. Description of an implantable platform permitting biofluid transfer through an implanted surface has been described, in part, previously in the U.S. patent application Ser. No. 10/032,765, now U.S. Publication No. US 2003-0004403 A1, hereby incorporated by reference in its entirety.

In one embodiment of the invention, the connection between the access port and the disposable section is a structure located at the exit point of the access port from the skin, e.g. a percutaneous mounting ring. In alternate embodiments, the connection structure is located at the end of a catheter-like tube, joining the percutaneous access port to one or more disposable sections. In preferred embodiments of the invention, such connections are not permanent, but rather allow removal and replacement of the disposable section.

In an embodiment, part of this automatic system includes the use of one or more sensors providing feedback, allowing for adjustment in drug delivery rate, volume or schedule, either automatically or upon outside command. In certain embodiments, the DDP receives instructions or information either directly or indirectly from biosensors mounted on or within the body of the subject. The use of such information permits the creation of a closed loop system, enabling automatic adjustment of the therapeutic agent in response to changes in body bioparameters.

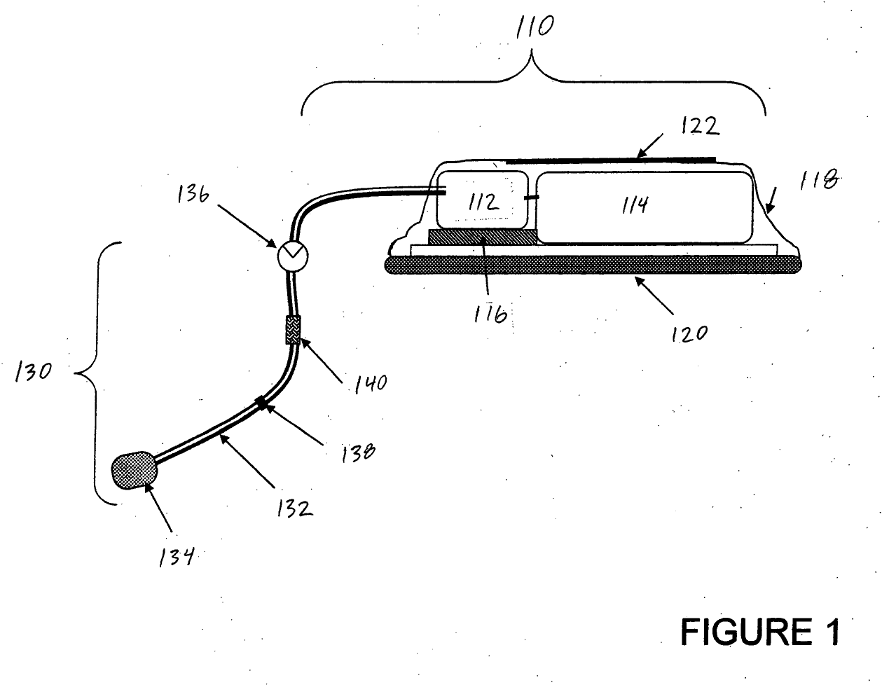

The invention generally relates to devices and apparatus for the automatic administration of therapeutic agents. An embodiment of the present invention is shown in FIG. 1. In this embodiment, a drug delivery platform 100 (DDP) is comprised of two primary sections, a disposable section 110 affixed to the skin (not shown), containing a pump 112, a drug reservoir 114, microcontrol circuitry and power source 116, and an access port 130 for the parenteral delivery of compounds received from the disposable section 110. The DDP may be used for the administration of therapeutic agents in a parenteral fashion.

A preferred embodiment of this invention is a device which automatically delivers therapeutic agents using an access port that has a percutaneous catheter-like tube 132. This delivery may be either continuous, periodic, or upon command. In a further refinement of this preferred embodiment, the catheter-like tube 132 has, at the distal (or implanted) terminus, an infusion structure 134 referred to as a biofluid head. An advantage of this embodiment is the use of a parenteral access device suitable for long-term implantation comprising the access port 130, to which one or more disposable sections 110 may be affixed in a successive fashion as the therapeutic agents employed are consumed or otherwise require replacement. Such a system avoids the need for repetitive penetrations of the skin in order to provide such parenteral access, yet provides flexibility in the amounts and administration schedules of said therapeutic agents.

In addition, by the automatic administration of the therapeutic agents offers multiple advantages over other methods of therapeutic administration, e.g. pills. These advantages include, but are not limited to, improving compliance with prescribed therapeutic agents, as well as improving data logging/recording of therapeutic agents taken and adjustments to dosages and regimens as well as of volumes delivered.

Access Port

The access port advantageously contains three principal elements in some embodiments: a) one or more parenteral fluid delivery locations present on at least one portion of the structure, e.g. a fluidic path to a least some bodily tissue from at least one lumenal space, b) one or more flexible tubing or catheter-like constructs having one or more lumenal spaces providing a fluidic passage within the access port; and c) one or more connection points outside the body wherein one or more catheter-like structures is joinable to at least one disposable section such that at least one fluidic communication, e.g. a fluidic pathway, may be established between the disposable section and at least one lumenal space within the access port. Additional elements may be present in various embodiments of the invention.

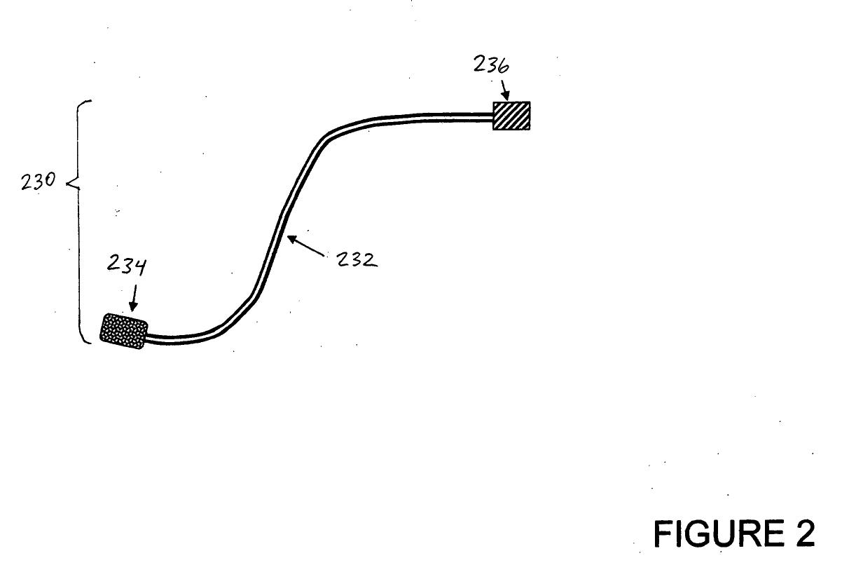

In a preferred embodiment of the invention, at least one fluid delivery location is at least partially rigid in nature and is termed the “biofluid head”. As seen in FIG. 2, in one embodiment of the invention, the biofluid head 234 resides at the distal terminus of the catheter-like tubing 232, which has at its proximal end a connector portion 236 for joining the access port 230 to a disposable section (not shown). The biofluid head 234 contains a plurality of openings through which the therapeutic agent may pass into the surrounding tissue.

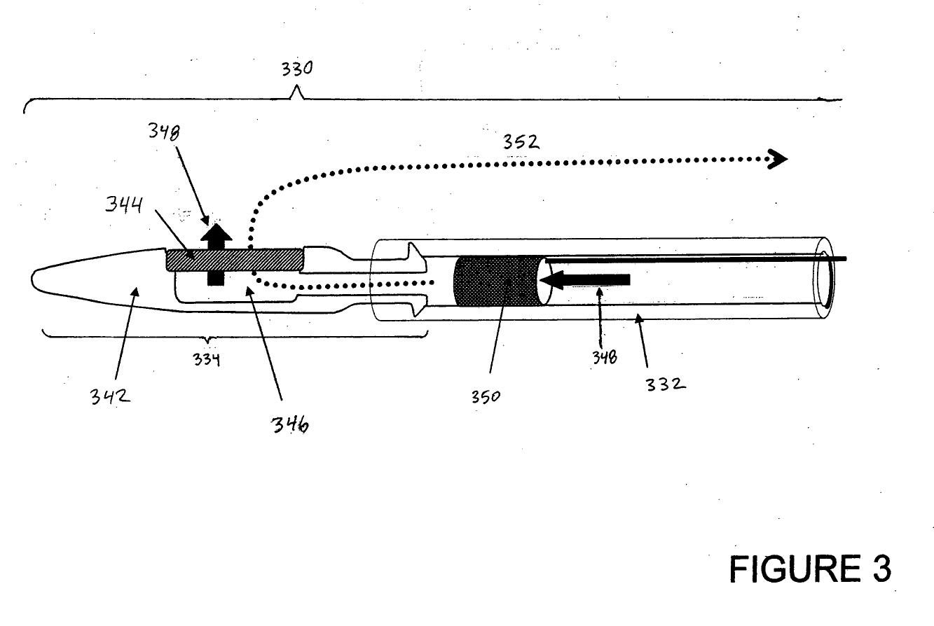

In one embodiment of the invention, the biofluid head may be comprised of a single assembly having both an outer surface and at least one inner surface describing at least one lumenal space within the biofluid head. To provide a fluidic path for therapeutic agent delivery to surrounding tissue, a plurality of holes extends from at least one interior lumenal space to an outer surface. The structure of the head may be comprised of one or more pieces with each piece comprised of one or more materials. One embodiment of a multipiece assembly is shown in FIG. 3.

FIG. 3 shows the distal, or implanted, end of an access port 330, in which the biofluid head structure 334 has two pieces. A first piece, referred to as the biofluid head body 342 comprises the body of the structure. There are no passages through the biofluid head body 342 between the interior lumenal space and the one outer surface. Positioned within the body is a biofluid head insert 344 having a plurality of passages, e.g. holes, permitting fluid passage from the interior lumen 346 of the biofluid head 334 to the outer surface of the biofluid head, as indicated by arrows 348. Other embodiments having one or more pieces are readily conceivable, e.g. in other embodiments of the invention the biofluid head itself may have a plurality of pieces and structures, and the embodiment shown in FIG. 3 is not intended to limit the scope of the invention.

In one embodiment of this invention, the cross-sectional dimension of these passages limits the ability of surrounding tissues and cells to migrate or invade into the lumenal space of the biofluid head, e.g. the cross-sectional dimension is generally less than 1 micron wide at the narrowest point of passage. In further embodiments, this cross sectional dimension is generally less than 250 nanometers at the narrowest point. Passages with such cross-sectional dimensions advantageously limit the infiltration of surrounding cells and are small enough to preclude the passage of any bacteria.

In one embodiment, the material of the biofluid head 334 having fluid passages, e.g. the insert 344, may be formed in whole or in part from one or more of a variety of biocompatible materials, including but not limited to: membranes, polymeric meshes, porous polymers, glass frits, microfabricated structures made from silicon or other materials commonly employed in semiconductor fabrication, or metals, e.g. titanium or stainless steel.

Various microfabricated structures and other possible structures or features which are configured to promote tissue ingrowth and prevent fibrous encapsulation of the biofluid head are discussed in U.S. patent application Ser. No. 10/984,681, filed on Nov. 8, 2004, hereby incorporated by reference in its entirety.

In embodiments in which the biofluid head comprises an insert which contains the fluid passages, the remainder of the biofluid head may be comprised of the same materials as the insert, or of different materials. The remainder of the biofluid head may be comprised of materials including, but not limited to, biocompatible plastics such as polyfluorinated polymers, polyetheretherketon (PEEK), silicones, or other rigid or semi-rigid materials such as glass, silicon, metals or metal alloys such as titanium or stainless steel.

In addition, in other embodiments, anticoagulation aids (e.g. heparin or other pharmaceutical anti-coagulants) may be present to prevent the adhesion of platelets or other clotting/rejection factors onto the biofluid head.

In yet other embodiments of the invention, the integration of the biofluid head 334 or portions of the biofluid head into the surrounding tissue may be desired in order to lessen encapsulation of the device by fibrous tissue as part of the body's rejection mechanism. In one embodiment, the surface may have structures or microfeatures having dimensions and topology promoting adherence of the surrounding cells (as opposed to initiating a rejection response including encapsulation and walling off of the implanted device.)

In addition, such embodiments may also include the use of one or more soft porous materials or layers on at least a portion of the outer surface of the biofluid head having properties encouraging surrounding tissue ingrowth. Such materials include, but are not limited to, hydrogels, polymeric gels or sponges such as polyvinyl alcohol-based polymers, or fibrous polymers comprised of naturally occurring or synthetic substances.

In yet other embodiments, the outer surface of the biofluid head employs one or more features which encourage surrounding tissue ingrowth and to minimize fibrous capsule formation. These features include, but are not limited to, coating the surface or portions of the surface with appropriate growth factors, adherence molecules and attractants, such as prothrombin activator, vitamin K, thrombin, fibrin, keratinocyte growth factor, activin, proteoglycans, cytokines, chemokines, TGF-beta, TNF-alpha, VEGF, PDGF, FGF, PAF, NGF, IL-4, IL-8, Insulin-like growth factor, integrins, laminin, fibronectin and other factors to promote the ingrowth of surrounding tissues.

In yet other embodiments of the invention, active features, such as the application of electrical currents may be utilized to minimize fibrous capsule encapsulation. Such features are understood to be useful for accelerating those processes associated with wound healing/fibroblast infiltration. In the context of this invention, such electric currents would be applied in a converse fashion, limiting fibroblast infiltration and therefore minimizing the amounts of collagen, which comprises a significant portion of the fibrous capsule deposited by such cell types. As seen in FIG. 3, one or more electrodes 350 for application of electric current 352 may be incorporated within the lumen 346, on or within other portions of the access port 330, or in positions adjacent to surfaces where minimization of capsule formation is desired. As seen in FIG. 1, one or more counter electrodes 138 to complete the current circuit through the tissue may be placed elsewhere on the access port 130, or within/on the tissue (skin) of the subject.

In still other embodiments of the invention, specialized biomedia can be incorporated into the biofluid and/or therapeutic agent delivery solution for the purpose of minimizing inflammation, infection, capsule formation or, alternatively, promoting surrounding tissue ingrowth and biofluid head biocompatibility. Such media may include factors including, but not limited to, glucocorticoids, antibiotics, bacteriostatic agents, proteases or growth factors, cytokines or nutrients.

Other embodiments include the use of microdevices, e.g. MEMS (microelectro-mechanical systems) or MOEMS (microoptoelectromechanical systems) microstructures, that remain sealed or otherwise in an “off” position, until activated. Upon activation (based upon received instruction), vias or passages may open up within the microdevice, resulting in micropassages into which extracellular fluid may flow. In yet other embodiments of the invention, micron scale “scrapers” within the microdevice may also be employed in conjunction with flushing to remove debris and gain access to surrounding tissue fluid. Additional approaches, e.g. the use of electrical, or photonic forces, or chemical agents, may also be employed to sweep biomolecules or other forms of cellular debris away from the passages, biofluid head and/or improve access port function.

All of the embodiments described above may be applied alone or in various combinations to provide improved biofluid head performance, dependent upon the overall device needs and the tissues into which the biofluid head is implanted.

As seen in FIG. 3, the biofluid head 342 is physically connected to a structure 332, shown here as a catheter-like tube, having one or more lumenal passages 346 through which therapeutic agents and/or other materials may be passed. In a preferred embodiment, this structure 332 is flexible, allowing curves or twists along its length dependent upon the forces applied, e.g. having a bend within its length due to the method and route of insertion. Such structures may be comprised from one or more materials and may be comprised of one or more layers or sections. Such catheter-like structures are preferably constructed from biocompatible materials, well known to those skilled in the art of catheters, and include, but are not limited to, polyurethanes, silicones, expanded forms of polytetrafluorethylenes, stainless steels, or other metal alloys. To provide additional mechanical strength, a laminate layer comprised of nylon or high-strength fiber mesh may be added, e.g. KEVLAR (a nylon laminate), which adds strength while maintaining the required flexibility. Flexibility and ductility are preferred characteristics for comfort and acceptance of this implant technology.

The catheter-like tubing may have one or more passages for the purpose of introducing one or more fluids into the biofluid head or for introducing or providing a pathway for mechanical, electrical or optical device/structure insertion, e.g. electrode or biosensor insertion. In other embodiments of the invention, one or more passages may provide a passage to allow biofluids to pass from the biofluid head and through the catheter-like tubing for the purpose of analyte sampling, or other diagnostic/therapeutic purposes.

In one or more embodiments of the invention, the catheter-like structure may incorporate one or more valve devices along the course of fluid passageways. Such structures may include, but are not limited to, ball valves, flaps or MEMS-type microstructures having mechanical, electrical or other types of control. Such valves may be useful for assuring the unidirectional flow of liquids within passages, e.g. limiting surrounding biofluid infiltration or limiting the passage of air or other undesired materials through the access port.

In one or more embodiments, those regions of the fluid passage structure (catheter-like tubing) beneath the surface of the skin may have one or more features to promote surrounding tissue ingrowth or other form of stabilization of the tubing structure with the surrounding tissue. Such stabilization is desirable to reduce mechanical motion of the implanted tubing within the tissue and thereby lessen trauma resultant from this motion. In addition, such stabilization may serve to limit the migration of bacteria or other noxious agents along the outer aspects of the tubing and into the body of the subject.

Embodiments of such stabilization features include the use of those features described previously with respect to the biofluid head to promote surrounding tissue ingrowth, e.g. microtexturing, or the presence of agents such as growth factors, adherence molecules and attractants. In addition, devices or materials such as ingrowth collars, made from materials such as Dacron cuffs, may be affixed to outer aspects of the catheter-like tubing to provide a method of anchoring the tubing into the surrounding tissue, either through the use of sutures or through tissue ingrowth. Such stabilization methods are well known to those skilled in the art of catheters.

In addition to the use of such stabilization features to promote surrounding tissue ingrowth onto the catheter-like structure, electric currents may be applied to enhance the deposition of collagen and other extracellular matrix proteins in the vicinity of the catheter-like tubing, particularly near stabilization structures such as an ingrowth collar. Such currents may advantageously result in the migration of fibroblasts towards an electrode having appropriate polarity. This is in contrast to the use of electric currents described with respect to the biofluid head, wherein the fibroblasts are guided away from the electrode. If the counter electrode for the biofluid head is positioned in the vicinity of the catheter-like tubing, e.g. beneath a porous ingrowth collar or structure, then upon activation of an electrode causing movement of fibroblasts and/or other cell types away from the biofluid head, fibroblasts will be attracted to the counter electrode positioned in the vicinity of the ingrowth collar. Thus, one current orientation and application may serve dual purposes: a reduction of capsule formation about the biofluid head and enhanced matrix deposition in the region of an ingrowth collar.

As can be seen in FIG. 2, upon exiting from the body (not shown), the catheter-like structure 232 is terminated on the proximal end by a connector portion 236. Such connector portions may include, but are not limited to, mounting rings affixed to the surface of the body or end fittings upon the proximal end of the tubing such as Luer Lock connections.

As can be seen in FIG. 1, such connector portions 136 are intended as an interface point between the access port 130 and the disposable section 110 and are intended for one or more connections to be made between the implanted access port and one or more disposable sections during the useful lifetime of the access port. Such connections permit the use of a long-term implanted access port and one or more disposable sections having shorter useful lifetimes. In addition, such connections are intended to provide a fluidic connection or pathway between the access port and the disposable section.

In other embodiments of the invention, such connector portions also provide electrical, optical or mechanical connections between the access port and one or more disposable sections. In embodiments in which the access port comprises one or more electrodes, connections may be provided between a power source in the disposable section and the electrodes in the access port. In addition, in further embodiments of the invention, the access port comprises one or more sensors in communication with controlling circuitry located in the disposable section, as is discussed in greater detail later. Connections may be provided between the sensors and the disposable section at the connection point, enabling the sensors to relay information to the controlling circuitry.

In still other embodiments of the invention, the connector portion or other elements within the access port contain information providing unique identification of the access port. This information may be optical, mechanical or electrical in nature. Such information may be relayed to controlling circuitry in the disposable section in either an automatic or manual fashion.

In certain embodiments of the invention, the connector portions also contain features to enable easy handling by the elderly or other individuals not having full manual dexterity. Such features may include, but are not limited to, enlarged sections or flanges to permit easy grasping, bright colors to permit ease of visualization, or audible or visual feedback systems indicating correct or incorrect connection between the access port and a disposable section.

In preferred embodiments of the present invention, and in contrast to the prior art discussed previously, the disposable portion of the DDP contains many of the more complex and costly components, particularly the pumping device, the power source, and at least some of the controlling circuitry. While the total cost of the device may be increased as a result of this, such an arrangement presents numerous advantages.

Because embodiments of the invention comprise a clinician implanted access port which is suitable for long term implantation (about 30 days or more), avoiding unnecessary complexity in the design of the access port will increase the reliability and longevity of the device because the presence of multiple components increases the overall likelihood of access port failure due to failure by at least one of these components. Failure of a component within the access port may necessitate replacement of the access port by a clinician, which may necessitate an additional trip to a clinician, and increase the overall cost to the patient. In addition, such a failure may cause a significant delay in the delivery of the therapeutic agent, due to the time required to have a clinician replace the access port. By placing more complex devices in the disposable portion, which in certain embodiments is readily replaceable by the user, the cost and hassle of replacement of non-working components, as well as the danger resulting from the failure of a component, are greatly reduced.

In addition, such an arrangement allows for additional flexibility in terms of the therapeutic agent to be delivered. As is discussed in greater detail later, various pumping devices may be employed in the delivery of therapeutic agents. Some pumping devices are better suited for delivery of certain therapeutic agents than others. When multiple therapeutic agents are to be delivered to a patient, embodiments of the present invention advantageously permit the use of a single access port for delivery of the multiple therapeutic agents by means of multiple pumping devices located in corresponding disposable sections. Such disposable sections may be connected to the access port either at the same time or in an alternating manner.

For instance, a physician can prescribe multiple courses of therapeutic agents to be administered via a DDP such that one course of a therapeutic agent is to be administered, followed by a course of a second therapeutic agent once the course of the first therapeutic agent has terminated. In doing so, the physician need not select two therapeutic agents which are capable of delivery via the same pumping device, because each therapeutic agent can be delivered via a different pumping device. Thus, a device according to a preferred embodiment of the present invention advantageously reduces limitations on the selection of therapeutic agents to be administered.

As discussed above, certain subjects may not have full manual dexterity. By reducing the complexity of the access port, the complexity of the connector portions can be reduced. In addition, in certain embodiments, the disposable section may be slightly larger than disposable portions of prior art devices, due to the additional components located within the disposable section, making it easier for persons without full dexterity to remove and replace disposable sections. Additionally, placing the controlling circuitry and transceiver circuitry in the same disposable section as the therapeutic agent to be delivered permits unique identification of each disposable section or of components or reagents within the disposable section.

In other embodiments of the invention, the connector portions (as well as other structures within the access port) may have other features, including, but not limited to, circuitry, antennae, a power source or a pumping device, that may aid in the function of the overall apparatus. By including such features within the access port, the overall cost of the apparatus may be lowered by not having to replace such features (components) with the replacement of each disposable section. However, for the reasons discussed above, inclusion of additional components in the access port will lessen or eliminate the advantages of the preferred embodiments. Inclusion of such components in the access port, particularly a pumping device or controlling circuitry, has a negative impact on the flexibility of the access port as a delivery port for a range of therapeutic agents, and may have a negative impact on the longevity and reliability of the implanted device.

To aid with the manufacture, storage, in-field calibration and insertion of the access port, a form of biocompatible hydrogel or similar substance may be used to coat or encapsulate the biofluid head. The catheter-like tubing may also be filled or coated with this hydrogel. The hydrogel may contain preservatives, anti-inflammatory agents, anticoagulants, bioactive agents, e.g. growth factors, cytokines or other bioactive agents, and antibiotics or antimicrobial agents. A form of hydrogel (e.g. select agarose gels, carrageenan gels, collagen gels, or other biocompatible synthetic or natural gels) may also be employed which exhibits the property of either being gel or liquid in nature in a temperature-dependent fashion. In particular, at or around room temperature the material has high viscosity and is gel-like in nature. When raised to body temperature, the material becomes fluid and is absorbed by the surrounding tissue. These hydrogel materials may be used alone or in conjunction with other forms of hydrogel or other previously described materials which provide a matrix for tissue ingrowth.

Disposable Section

An embodiment of the invention having a disposable section is shown in FIG. 1. The disposable section 110 has one or more containment areas 114 containing one or more therapeutic agents to be parenterally administered to a subject, a pumping device 112 for delivery of such agents, a power source and controlling circuitry 116 to regulate the administration of such agents, for said circuitry and pump, and an adhesive portion 120 for affixing the disposable section 110 onto the body of a subject.

In various embodiments of the invention, the disposable section 110 may operate in an autonomous or fully contained fashion, or it may dispense therapeutic agents in response to instructions received either directly from an input device (not shown), which may be located on the disposable section, or indirectly received through wireless communication with the disposable section. In this latter embodiment, the disposable section comprises additional communication features, such as transceiver circuitry (not shown) and an antenna 122, for said indirect communication, e.g. through a LAN network. The disposable section can download information to a receiving station or a display either automatically, or upon command. This downloading may be done either continually, or on a periodic basis, depending on factors such as battery life and the need to continually monitor the information. The information downloaded may be information which was stored on the DDP or relayed to the DDP from elsewhere, and this information may be converted, such as a processed signal from a sensor, or encrypted.

In still other embodiments of the invention, the communication aspects of the DDP (whether contained entirely or in part within the disposable section or access port) also may be able to relay or transmit other wireless communications from other DDPs or from other devices or instruments, e.g. from implanted diagnostic systems.

Pumping devices are well known to those skilled in the art of ambulatory pumping systems. Such pumping devices may possibly include but are not limited to: fluid pumps, e.g. syringe type pumps, electrochemical pumps, mechanical (spring) pumps, or MEMS-based micromachined devices; mechanical (manual) pumping; chemical reactions, e.g. production of gases or pressure to aid delivery; or electrical pumping, e.g. ionophoretic transport. In certain embodiments, the pumping devices may include valving or metering devices to aid in the regulation of therapeutic drug fluid delivery. In yet other embodiments of the invention, electric fields may be employed to aid in the delivery of therapeutic agents, e.g. through ionophoresis or electroosmostic activities.

In embodiments of the invention, the fluid path from the pumping and reservoir devices may also include one or more filtering features to limit the passage of bacteria or other undesired elements from passing from the disposable section into the access port lumenal space.

Therapeutic agents may include, but are not limited to, small molecules, peptides, proteins, or modified proteins. Examples of such agents include, but are not limited to, cardiovascular agents (e.g. b-type natriuretic peptides (BNP), trepostinil sodium, beta blockers, calcium channel blockers, vasopressin antagonists, cAMP enhancing agents, endothelin receptor antagonists, digoxin, inotropes, nitrates, prostacyclins including Remoduling and nitroglycerin), angiotensin II converting enzyme inhibitors and angiotensin antagonists, loop diuretics (e.g. furosemide), thiazides and other diuretics (e.g. specific aldosterone receptor antagonists, spironolactone), phosphodiesterase inhibitors, calcium sensitizers, adrenergic agents, advanced glycosylation endproduct crosslink breaker (e.g. ALT-711), xanthine oxidase inhibitors (e.g. allopurinol), cytokines and hormones, chemotherapeutic agents, pain management agents, blood cell proliferation agents (e.g. erythropoietin), antibodies, antibiotics, antiviral agents, immunosuppressants, vitamins, antioxidants, anti-inflammatory agents, anticoagulation agents (e.g. warfarin), agents for the treatment of (e.g. insulin, pramlintide acetate), and antipsychotic or behavior modification agents, (e.g. methylphenidate). Therapeutic agents may also include deliver of materials such as eukaryotic or prokaryotic cells, e.g. stem cells, gene modification tools, e.g. genetically altered viruses, or nanoscale materials and devices.

The therapeutic agents to be delivered may be mixed with additional fluids or reagents, e.g. water, physiological compatible buffers and components, dimethyl sulfoxide or other solvents, to facilitate generation of active materials or the absorption or uptake of the materials, compounds, etc. by the measured subject. Once added, the delivery system may signal the controlling circuitry as to the addition of the compounds, materials or devices or the addition may be monitored by sensors detecting either the agents directly or indirectly through measured bioparameters or other sensing methods.

In addition, one or more materials or agents may be delivered in addition to one or more therapeutic agents to promote acceptance of the Access Port by the user and to maximize device lifetime. These materials may include, but are not limited to, local anesthetics, bacteriostatic agents, pH or other physical environment modifying agents, or local inflammatory response control agents.

The therapeutic agents to be administered may be stored within reservoirs or other containment methods within the disposable section. The therapeutic agents may be stored in either biologically active or inactive states. The storage form may include aerosols; compressed gases; liquid storage, e.g. suspensions, solutions or gels; and/or dry forms of storage, e.g. powder, granules or films. The reservoir container may have additional features to enhance therapeutic agent or material stability. These features may include, but are not limited to, bacteriostatic agents, e.g. leeching of trace agents from the wall to limit bacterial growth, and physical environment modulation such as temperature control and ambient light shielding.

In certain embodiments of the invention, mechanical flushing of the biofluid head may be desired to clear the fluid passages. Flushing can be performed either manually by the user, or automatically through the use of channels or compartments which release saline or other physiologically compatible solution upon the sensing of occlusion, rejection or other factors which may diminish the intended performance of the device. In such embodiments, reservoirs for the flushing agent may be different than those employed for the therapeutic agents. In addition, the lumenal space utilized in the catheter-like tubing may be the same or different than that used for passage of the therapeutic agent.

In various embodiments, controlling circuitry may control activities of the pumping devices and communication features, and may control input/assessment of input from sensors. These sensors may include, but are not limited to, sensors gauging system performance and sensors associated with detection of physiological parameters, e.g. bioanalytes or physical measurements such as temperature. In embodiments having feedback from physiological parameters (whether as part of the DDP or from diagnostic devices external to the DDP), closed loop therapeutic delivery based upon said sensor input is enabled and may employ in part or in whole controlling circuitry contained within the disposable section.

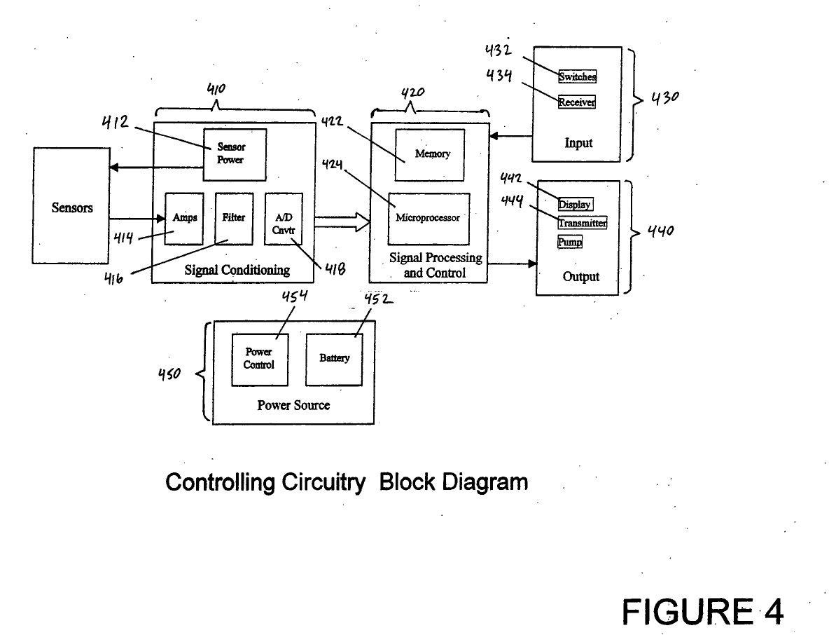

A block diagram illustrating functions of the controlling circuitry in an embodiment of the invention is shown in FIG. 4. As can be seen from the figure, functions contained within the controlling circuitry may include, but are not limited to, signal conditioning 410, signal processing and control 420, input 430, and output 440. Signal conditioning converts the analog sensor output to a digital signal. In further embodiments, the controlling circuitry may include electronic circuits that drive sensors (sensor power source 412), amplify and process the sensor outputs (amplifier 414 and filter 416), and convert these “conditioned” sensor outputs to a digital signal (A/D Converter 418). Signal processing and control converts the digitized sensor output to useful information. It generally includes a microprocessor 422, memory 424, and a software program (firmware, not shown) necessary to control the operation of the microprocessor. Inputs and Outputs (I/O) may be contained on the disposable section itself, possibly including but not limited to, switches 432, input keys (not shown), and displays 442, or located remotely.

In those embodiments of the invention employing remote I/O, a method of wireless communication (Receiver 434 and Transmitter 444) may be employed to communicate with a remote I/O device. This communication may or may not be encrypted for data security. In a preferred embodiment of the invention, wireless communication is encrypted. In addition, wireless communication may also be bi-directional to acknowledge successful receipt of transmission and to change the monitoring criteria (monitored parameters, delivery periods, etc.). Communication may continue beyond the remote I/O device through the use of secondary communication to, for example, a central data management system.

For cost, size and reliability reasons, in certain embodiments of the invention, as much of the above circuitry as possible is integrated onto a single integrated circuit. This may include all or portions of signal conditioning, signal processing and control, power control, transmitter and receiver.

As noted above, in certain embodiments of the invention, sensors may be included within the DDP or other devices affixed or implanted within the subject or otherwise obtaining measurements from the subject. Sensors may be electrical, chemical/bio-chemical, mechanical or any other device that converts a physiological parameter to an electrical or other form of readable signal. Such signals may provide input data used for adjusting therapeutic drug delivery. Table 1 shows exemplary physiological parameters that may be monitored and associated preferred sensing methods, but is not intended to limit the range of parameters which can be measured in embodiments of the present invention, or the sensing methods which can be utilized.

TABLE 1
Potential Physiological Parameters Providing
Data for Adjusting Therapeutic Drug Delivery

| Parameter | Preferred Sensing Method |
|---|---|
| Blood Pressure | pressure transducer, pulse |
| | propagation time |
| Subject Temperature | thermistor, silicon junction, |
| | thermocouple |
| Heart Rate | ECG analysis, pressure, reflectance |
| Kilocalorie Expenditure | algorithm based on heart rate & |
| | data input (e.g. height, weight, |
| | sex) |
| Respiration | accelerometer, impedance |
| ECG waveforms | multiple electrodes |
| ECG intervals | ECG waveform analysis |
| Blood oxygen | optical analysis |
| Body water (segmental or total) | impedance |
| Body metabolites, hormones, etc. | enzyme-linked impedance or voltage, |
| (e.g. glucose, BNP, serotonin, | ion selective electrodes |
| Na⁺) | |

Additional sensors may include those devices for sensing pressure, clarity or other measures of DDP performance, including the status of the therapeutic agents within the containment areas or delivery performance.

In certain embodiments of the present invention, one or more sensors may be located in, or partially extend into, the access port. Although it will be desirable, in certain applications, to minimize the amount of complex circuitry located in the access port in order to provide the advantages discussed previously, certain types of sensors require implantation within the body of a subject. In an embodiment in which a sensor, such as one configured to provide information regarding blood oxygen, is located within the access port and the DDP controlling circuitry is located in the disposable section, the connection point may provide not only a fluid connection between the two portions of the DDP, but also a connection which will permit sensor information to travel between the sensor and the controlling circuitry. As noted previously, this connection may be optical, electrical, mechanical, or of any other type suitable for conveying information between a sensor and the controlling circuitry. In alternate embodiments, this communication between the sensor and the controlling circuitry may be wireless communication.

A power source may be necessary to enable the electronic circuitry, the pumping device and in certain embodiments of the invention, the electrical currents applied to the access port. As seen in FIG. 4, the power source 450 generally includes an electrical source of power, e.g. a battery 452, and circuits that condition the battery output (voltage and/or current regulation) and maximize battery life (Power Control Circuitry 454). Power may also be inductively coupled to the DDP or be supplied through direct or indirect methods such as, but not limited to, responder (RF) technology, photonic technology (photovoltaic cells), the subject's own energy, e.g. motion, internal chemistry, including ATP molecules, glucose, or other energy supplying compounds, or osmotic pressure.

In an embodiment in which a power-requiring component is located within the access port, the connection point between the access port and the disposable section may include a connection which provides power to the power-requiring component from the power source located within the disposable section. A separate power source located within the access port, such as an implanted battery, may also be used to provide power to the power-requiring component, and would reduce the complexity of the connection points, but in applications in which the component requires a significant amount of power, providing a power source within the disposable section may increase the amount of time during which the access port can remain implanted, as there is no battery within the access port which requires replacement. In addition, the size of the access port is advantageously kept to a minimum.

Methods to attach the disposable section onto a subject, e.g. on the skin, include, but are not limited to, use of adhesives (as seen in FIG. 1), tapes or straps, such that a position of the disposable section may remain fixed to a certain location of the body throughout the useful period of the disposable section. In certain embodiments of the invention, a length of the catheter-like tubing extends from the opening in the skin for a length allowing successive placement of two or more disposable sections on different locations on the subject's skin surface such that the skin surface is allowed to recover from the application of adhesive or other method of fastening before that same region of skin surface has another disposable section affixed to it.

As shown in FIG. 1, the outer surface 118 of the disposable section 110 may be comprised of one or more layers, including layer(s) possibly containing electronic components, (e.g. antenna 122, visual or audible display), sensors (e.g. temperature, pressure transducers, not shown) or input devices (buttons, switches, not shown). In a preferred embodiment of the invention, the outer surface 118 of the disposable section is substantially water resistant to allow use of the DDP in a variety of environments, e.g. showering or exercise, where water may be encountered.

Operation of Drug Delivery Platform

In one preferred mode of operation of the DDP, the access port is installed by a clinician using a trocar like tool such that the distal end resides in a subcutaneous location within a subject's body. A first disposable section is affixed to the subject and connected to the access port. Activation of the platform using the circuitry of the disposable section is performed upon connection. Such activation may include, but is not limited to, verification that the connection to the access port has been accomplished, the beginning of therapeutic agent delivery according to included instructions and transmittal of the information that the DDP has been activated, the nature of the therapeutic agent being delivered and schedule of delivery. Such information may be transmitted via a LAN to a local display/data input device and/or further transmitted to a remote data management system for logging and outside review.

Upon outside review, instructions may be remotely inputted into the disposable section to adjust the delivery of the therapeutic agents, e.g. rate, schedule or volumes. Such instructions may be in response to values or parameters received from sensors located either on the disposable section or from other diagnostic devices. When it is desirable to replace the first disposable section, e.g. the reservoir is depleted, following a defined period of use, or upon the need to switch medications, the first disposable section is removed and replaced by a second disposable section containing fresh therapeutic agent to be delivered. Again, activation of this second disposable section occurs in a fashion akin to that of the first.

All of the embodiments of the invention described above may be applied alone or in various combinations to provide therapeutic drug delivery. One of ordinary skill will readily understand that numerous permutations of the invention are conceivable and the embodiments described above are not intended to limit the scope of the invention.

While the above detailed description has shown, described and pointed out the fundamental novel features of the invention as applied to various embodiments, it will be understood that various omissions and substitutions and changes in the form and details of the system illustrated may be made by those skilled in the art, without departing from the intent of the invention. The foregoing description details certain embodiments of the invention. It will be appreciated, however, that no matter how detailed the foregoing appears, the invention may be embodied in other specific forms without departing from its spirit or essential characteristics. The described embodiment is to be considered in all respects only as illustrative and not restrictive and the scope of the invention is, therefore, indicated by the appended claims rather than by the foregoing description. All changes which come within the meaning and range of equivalency of the claims are to be embraced within their scope.

1. A parenteral therapeutic agent delivery device comprising:
an access port comprising a parenteral fluid delivery location, an interior lumenal space, and a first connection point;
a disposable section, configured for attachment to the body of a subject, comprising a reservoir configured to hold a therapeutic agent, a pumping device, controlling circuitry to regulate delivery of the therapeutic agent, and a second connection point configured to mate with said first connection point.

2. The device of claim 1, wherein the disposable section additionally comprises transceiver circuitry, an antenna, and a power source.

3. The device of claim 2, wherein the controlling circuitry is configured to utilize signals received via the antenna and the transceiver circuitry in regulating the delivery of the therapeutic agent.

4. The device of claim 1, wherein the disposable section additionally comprises an input device.

5. The device of claim 1, wherein the controlling circuitry is configured to utilize signals from the input device in regulating delivery of the therapeutic agent.

6. The device of claim 1, wherein the device additionally comprises sensors.

7. The device of claim 6, wherein the controlling circuitry is configured to process signals received from the sensors.

8. The device of claim 7, wherein the controlling circuitry is configured to utilize processed signals from the sensors in regulating the delivery of the therapeutic agent.

9. The device of claim 7, wherein the disposable section additionally comprises transceiver circuitry and an antenna, and wherein the controlling circuitry is configured to relay processed signals to an external device via the transceiver circuitry and antenna.

10. The device of claim 2, wherein the controlling circuitry is configured to transmit information regarding the delivery of the therapeutic agent via the transceiver circuitry and antenna.

11. A parenteral fluid delivery device comprising:
an access port comprising a first connection point, a lumenal space in fluid communication with the first connection point, and a biofluid head, said biofluid head configured for long term implantation by incorporating features promoting cellular ingrowth and inhibiting fibrous encapsulation of at least a portion of the biofluid head; and
a disposable section, configured for attachment to the body of a subject, comprising a reservoir configured to hold fluid, a pumping device, controlling circuitry to regulate delivery of the fluid, and a second connection point.

12. The device of claim 11, said biofluid head comprising a plurality of passages extending from the lumenal space to an exterior surface of the biofluid head.

13. The device of claim 12, said biofluid head comprising an insert structure comprising said plurality of passages.

14. The device of claim 12, wherein the passages have a cross-sectional dimension which limits the ability of surrounding tissues and cells to enter the lumenal space.

15. The device of claim 12, wherein the plurality of passages have a cross-sectional dimension of less than about one micron at a point along their length.

16. The device of claim 12, wherein the plurality of passages have a cross-sectional dimension of less than about 250 nanometers at a point along their length.

17. The device of claim 12, wherein at least a portion of the exterior of the biofluid head has features intended to reduce fibrous encapsulation of at least said portion of the biofluid head.

18. The device of claim 11, said access port comprising a first electrode located at a point near said biofluid head, wherein said first electrode is configured to generate a current in conjunction with a counter electrode such that fibrous encapsulation of the region of the biofluid head close to the first electrode is minimized.

19. The device of claim 18, the access port additionally comprising a counter electrode, said first and counter electrodes being configured to generate an electric current such that movement of cells toward the region of the access port close to the counter electrode is increased.

20. The device of claim 19, said access port additionally comprising stabilization feature, and wherein said counter electrode is located near said stabilization feature.

21. The device of claim 20, wherein said stabilization feature comprises an ingrowth collar.

22. The device of claim 11, wherein said biofluid head is configured for implantation for 30 days or more.

23. The device of claim 11, wherein said biofluid head is configured for implantation for 90 days or more.

24. A parenteral fluid delivery device comprising:
an implant portion suitable for long-term implantation, comprising a parenteral fluid delivery location, a catheter-like construct defining a lumen, and a first connection point; and
a disposable portion, configured for attachment to the body of a subject, comprising a reservoir configured to hold fluid, a pumping device, controlling circuitry to regulate the release of the fluid, and a second connection point configured to detachably mate with said first connection point.

25. The device of claim 4, additionally comprising a sensor, wherein the controlling circuitry is configured to process signals received from the sensor and utilize said processed signals in the regulation of the release of the fluid.

26. The device of claim 24, wherein the implant portion additionally comprises a stabilization feature.

27. The device of claim 26, wherein the stabilization feature comprises an ingrowth collar.

28. The device of claim 24, wherein said access port additionally comprises a first electrode, configured to generate an electric current in conjunction with a counter electrode such that movement of cells toward the region of the access port close to the counter electrode is increased.

29. The device of claim 24, wherein said implant portion is configured for implantation for 30 days or more.

30. The device of claim 24, wherein said implant portion is configured for implantation for 90 days or more.
